(19) United States
(12) Patent Application Publication
(10) Pub. No.: US 2012/0179053 A1
(43) Pub. Date: Jul. 12, 2012

(54) APPARATUS FOR MEASURING A PROPAGATION VELOCITY OF A BLOOD PRESSURE WAVE

(75) Inventors: Vincenzo Gemignani, Torre del Lago Puccini (IT); Francesco Faita, La Spezia (IT); Elisabetta Bianchini, Saltocchio - Lucca (IT)

(73) Assignees: FONDAZIONE TOSCANA GABRIELE MONASTERIO, Pisa (IT); CNR-DIPARTIMENTO DI MEDICINA, Roma (IT)

(21) Appl. No.: 13/388,098

(22) PCT Filed: Aug. 2, 2010
(86) PCT No.: PCT/IB10/01901

§ 371 Date: Mar. 20, 2012

(30) Foreign Application Priority Data
Jul. 31, 2009 (IT) PI2009A000099

(51) Int. Cl. A61B 5/021

(52) U.S. Cl. 600/485

(57) ABSTRACT

An apparatus for measuring the propagation velocity of a pressure wave comprises a first sensor of cutaneous vibration to measure a vibration generated in a first application point, creating a corresponding first signal, and a second sensor of cutaneous vibrations to measure a local cutaneous vibration generated in second point of an arterial vessel, creating a corresponding second signal caused by the deformation of the vessel responsive to the progression of the pressure wave in the vessel. A control unit detects on the first and second signal respectively a first instant time T1 and a second instant time T2 corresponding to a same event of a cardiac cycle. On the basis of T1 and T2 a transit time PTT (Pulse Transit Time) is calculated of the pressure wave and then the propagation velocity is measured of the pressure wave as the ratio between the length of the path arterial.

### FIELD OF THE INVENTION

The present invention relates to an apparatus for measuring the propagation velocity of a pressure wave in the central arterial system, by detecting signals of the cutaneous vibrations generated by the heart and signals of the cutaneous vibrations generated by the movement of the blood in an artery.

Furthermore, the above described apparatus is adapted to effect a measurement at different points of a same arterial vessel.

Furthermore, the invention relates to an apparatus for measuring variation of the propagation velocity of a pressure wave in the central arterial system.

### DESCRIPTION OF THE PRIOR ART

As well known, increasing the stiffness of blood vessels is a new and early index of increased cardiovascular risk. Different techniques exist for determining the arterial stiffness at systemic, regional and local levels. In particular, the regional arterial stiffness, i.e. the stiffness measurement in a determined portion of an artery, can be evaluated by measurement of the propagation velocity of the pressure wave also called PWV “Pulse Wave Velocity”, considered as a technique of reference in this field.

Recently, in fact, it has been shown that the parameter of aortic stiffness determined by the PWV technique is an independent predictor of cardiovascular events in patients at high risk and in the general population.

Presently, different methods have been developed for determining the PWV: by means of sonography at high spatial resolution, as described in Pannier B, Avolio A P, Hoeks A, Mancia G, Takazawa K,—“Methods and devices for measuring arterial compliance in humans”—Am J Hypertens 2002;15:743-753; or Doppler sonography, as in Lehmann E D, Hopkins K D, Rawesh A, Joseph R C, Kongroove K, Coppack S W, Gosling R, “Relation between number of cardiovascular risk factors/events and noninvasive doppler ultrasound assessments of aortic compliance”—Hypertension 1998;32:565-569.

Other techniques provide, instead, the use of mechanical-transducers as in Asmar R, Benetos A, Topouchian J, Laurent P, Pannier B, Brisac A M, Target R, Levy B, “Assessment of the arterial distensibility by automatic pulse wave velocity measurement. Validation and clinical application studies” Hypertension 1995;26:485-490; or of tonometry, such as in Karamanoglu M, Gallagher D E, Avolio A P, O'Rourke MF—“Pressure wave propagation in to multibranched model of the human upper limb”—Am J Physiol 1995;269:H1363-H1369.

In particular, tonometry is the most diffused technique, since it is considered easy and reliable. In fact, by means of tonometry, the pressure wave is detected in two sites of interest, normally the carotid artery and the femoral artery, and combined with an ECG signal. In this case, the PWV is determined as the ratio between the duration or transit time called PTT “Pulse Transit Time” between the trough of the two waves and the distance between the two measurement sites. Even if largely used, this technique is affected by some limits owing to problems of estimating the distance between the measurement sites as well as difficulty of detecting the pressure wave in the femoral artery in obese subject and possible overestimation of the PTT in the presence of stenosis, i.e. a disease that involves narrowing of a duct, in this case a blood vessel.

New approaches for measuring the PWV have been found recently. For example in De Melis M, Morbiducci U, Scalise L, Tomasini E P, Delbeke D, Baets R, Van Bortel L M, Segers P—“A noncontact approach for evaluation of large artery stiffness: a preliminary study—Am J Hypertens—2008 December; 21(12):1280-3 the authors introduce a technique, based on the use of a laser, called “Optical Vibrocardiography”, for evaluating the PTT by the contemporaneous detection of the movement of the neck and of the inguen.

Even more recently, in US2009030328 a method has been proposed for calculating the PWV based on a pressure measurement in a single arterial site starting from which, by means of mathematical control of the signal, it is possible to evaluate the instants of arrival of an anterograde and retrograde wave and then the PWV same.

Other approaches, as disclosed in US2008275351 and in Hermeling E, Reesink K D, Reneman R S, Hoeks A P, Ultrasound Med Biol—“Measurement of local Pulse Wave Velocity: effects of signal processing on precision”—2007 May;33(5):774-81 are based, instead, on the control of sonographic images of a portion of artery. In particular, such techniques are based on the hypothesis of estimating displacements of the edge of the vessel with a frequency and precision to distinguish the progression of the pressure wave between a proximal portion and a distal portion of the vessel examined.

It is relevant to note that the PWV provides an estimation of the arterial stiffness of a vascular region defined by the actual position of the sensors used for the measurement. However, from a diagnostic viewpoint, it is known that it is very relevant to know the arterial stiffness of the vascular central portion, for example between heart and carotid artery, excluding thus the contribution of the peripheral muscular arteries.

A limit of this method is that the measurement of the pressure wave in an artery has some practical difficulty.

The problem has been faced in EP1338242 that introduces a mechanical solution to support a probe.

Other apparatus provide the measurement of the stiffness, responsive to, on the one hand, the detection of the heart tones, by means of phonometry, and of the pressure wave in a remote arterial site by a variety of types of sensor types, among which tonometry as disclosed in KR20020013820, or imaging as disclosed in EP1334694, or sphygmomanometry as disclosed in EP1302165. This way, they identify the PTT as the time period between the second heart tone and the “Dicrotic Notch” of the pulse wave, i.e. a small deflection observable in the decreasing portion of the shape of the pressure wave. One of the problems of these systems is the difficulty of a continuous and long monitoring.

Therefore, none of these apparatus offers a solution that is operatively easy, and is adapted to a continuous monitoring lasting several minutes, as required in certain diagnostic examinations such as, for example, pharmacological stress test and physical stress test.

In US2003220577, the use is described of phonometry for measuring the cardiac sounds both centrally, at the heart, both at a remote site. In this case, the sounds that are recorded distally are the effect of the propagation of the cardiac tones along the tissues, among which there are the bone structures and the vascular system, and their delay is then considered as due at a propagation velocity of the sound in the material, which is much higher than the PWV. The drawback of this system is that the propagation of the cardiac tones along the tissues is attenuated very quickly with the distance, and consequently the remote site has to be very close to the heart, and in any case the sonic signal to analyze is very weak and mistakable with the background noise.

In EP1249203, a microphone located at the heart and an arterial pressure sensor at an artery are used. In particular, the pressure sensor is mounted on the neck of the patient and is held in contact with the skin with a predetermined pressure, thus measuring the pressure wave of the arterial vessel, in particular of the carotid artery. In this case, then, the sensor detects a pressure at the artery. The use of a pressure sensor causes some drawbacks. Firstly, the pressure sensor has to be kept still, since a minimum movement affects the detection. To this end, in fact, special collars are used that the patient must wear during the time measuring step. These tools are however very uncomfortable for the patient same and not completely effective if the patient effects its normal activities.

In addition, the sensor mounted on the skin at the artery, produces a pressure that affects the hemodynamics of the blood flow. In fact, the application of the sensor causes a shrinkage of the cross section of the arterial vessel perturbing the normal dynamic behavior of the blood and altering the measurement. In other words, the pressure sensor obstructs partially the arterial vessel causing the pressure wave to hit against the obstructed zone and to transmit the signal to the pressure sensor. This causes changing the shape same of the arterial vessel, in addition to changing the hemodynamics, with subsequent further alteration of the measure. Furthermore, the pressure sensor is of difficult application since for keeping enough pressure on the vessel it is necessary the presence of an operator or the use of belts or collars of difficult application which tie the patient to stay still. In particular, the pressure of the operator cannot be steady and causes transmitting to the pressure sensor a noise generated by a wrong application. In case of the belt or collar, a small movement the patient can moving the sensor and affect the measurement. Furthermore, in certain zones of the body, such as on the neck, a belt is of difficult application.

It is felt, therefore, the need to provide a not invasive apparatus that is adapted to carry out a direct measurement on the arterial segment close to the heart to determine the real central PWV, not influenced by the presence of muscular arteries and then capable of providing more reliable clinic indications.

Furthermore, the need is felt measuring the PWV between two points of the arterial system different from the heart, measurement that is not provided by the presently existing systems.

### SUMMARY OF THE INVENTION

It is therefore a feature of the present invention to provide an apparatus for measuring the propagation velocity of the arterial system pressure wave avoiding the drawbacks of the apparatus of the prior art.

It is a particular feature of the present invention to provide an apparatus that provides a measurement of the propagation velocity of the wave pressure in the central arterial system, i.e. in the portion of the arterial system nearest to the heart.

It is a further particular feature of the present invention to provide an apparatus capable of measuring the propagation velocity of the pressure wave of the arterial system in a simple way, exceeding the difficulty of measuring the local pressure wave in the arterial vessels, such as carotid artery and femoral artery.

It is still a further feature of the present invention to provide an apparatus capable of monitoring precisely and in a substantially continuous way variation of the propagation velocity of the pressure wave of the arterial system during diagnostic examinations such as, for example, pharmacological stress test, physical stress test and stress test of desired other nature.

These and other objects are achieved through an apparatus for measuring the propagation velocity of a pressure wave in a cardiovascular system, in particular a pressure wave in a central arterial system, said apparatus comprising:
a first sensor of cutaneous vibrations, which is adapted to be mounted at a first application point of said arterial system for measuring a first cutaneous vibration, said first sensor creating a corresponding first cutaneous vibration signal;
a second sensor of cutaneous vibrations that is adapted to be mounted at a second application point of said arterial system different from said first point for measuring a second cutaneous vibration, said second sensor creating a corresponding second cutaneous vibration signal;
a means for determining the distance between said first and said second application points;
a control unit, said control unit comprising:
   a means for detecting said first and said second cutaneous vibration signals as input towards said control unit;
   a program means for calculating the propagation velocity of said pressure wave responsive to said distance between said first and said second application points and to said first and second cutaneous vibration signals, wherein said first and second sensors of cutaneous vibrations are adapted to measure vibrations set between 1 Hz and 20 KHz, in particular between 5 Hz and 80 Hz.

In particular, said first application point of the central arterial system of said first sensor of cutaneous vibrations is at the heart, in order to measure the vibration generated by the heartbeat, whereas said second application point of said second sensor of cutaneous vibrations is at an arterial vessel, in order to measure the vibration caused by the deformation of the vessel responsive to the movement of said pressure wave. In particular, both sensors of vibration are applied in a light contact with the skin of the patient without applying any pressure. This is particularly relevant especially at the arterial vessels since the adoption of cutaneous sensors makes it possible to eliminate measurement errors due to a pressure application to the vessels. This way, in fact, it is avoided that, at the application point of the sensor, there is a restriction of the cross section and of the shape of the arterial vessel.

Alternatively, said first and second local application points of said first and second sensors of cutaneous vibrations are at a same arterial vessel at a predetermined distance from each other, in order to measure a local cutaneous vibration by the deformation of the vessel in each of said points located on said same arterial vessel. This way, the sensors applied without any pressure to the same arterial vessel do not alter the hemodynamics of the blood flow and provide a careful and precise measurement of the pressure wave velocity.

In particular, said first and second sensors of cutaneous vibrations are applied by means of sticking plasters or other medical adhesives, in order to provide a light contact with the skin of the patient at the selected application point. This way, the first and second sensors do not affect the hemodynamics of the blood stream at their respective application points and the shape of the vessel, thus unaffecting the measurement of the pressure wave velocity.

Advantageously, said program means causes:
on said first cutaneous vibration signal a first instant time corresponding to a predetermined event of a cardiac cycle;
on said second signal a second instant time corresponding to the occurrence of the same event of the cardiac cycle as a local deformation of said arterial vessel;
a transit time of said pressure wave as a time period difference between said first and said second instant times;
   and calculates said propagation velocity of said pressure wave as the ratio between the distance between the heart and the application point of said second sensor and said transit time.

In particular, said second sensor detects the vibration caused by the deformation of the vessel responsive to the movement of said pressure wave.

This way, it is possible to apply the second sensor for a long time on the patient's skin, at the application point of the sensor near the arterial vessel, without any discomfort for the patient, and measuring continuously the second cutaneous vibration signal. The propagation velocity of said pressure wave, i.e. the PWV, is then calculated continuously, and a chart can be produced during diagnostic examinations such as, for example, a stress test.

Furthermore, instantaneous value of the PWV has a precision at least comparable to that obtainable with known systems.

Advantageously, said apparatus comprises acquisition means of an electrocardiographic signal, said electrocardiographic signal being used as synchronism time for determining said first and second instant times.

Preferably, said first sensor for acquisition of the signal of the cutaneous vibrations generated by the heart is located on the sternum whereas said second sensor for acquisition of the signal of the cutaneous vibrations generated by an arterial vessel is located on the neck of the patient.

In particular, said arterial vessel is the carotid artery.

Preferably, said first and second sensors are selected from the group comprised of: an accelerometer, a microphone, or an inertial sensor.

Preferably, said first instant time corresponds to closing the aortic valve whereas said second instant time corresponds to the “Dicrotic Notch” of the waveform of the diameter.

Alternatively, said first instant time corresponds to opening the aortic valve whereas said second instant time corresponds to start of a quick increase of the diameter, i.e. the wave trough. Such quick increase of diameter is due to arrival of the pulse pressure.

Advantageously, starting from said propagation velocity of the pressure wave said program means calculates other indexes of vascular stiffness, such as distensibility and Young modulus.

According to another aspect of the present invention said program means is adapted to cause:
in conditions that are prior to the imposition to the patient of a physical or pharmacological stress, or basal conditions, delay time Tₒ between a tone of said first signal and the corresponding tone of said second signal;
in conditions contemporaneous or following to the imposition to the patient of a physical or pharmacological stress, or post-basal conditions, delay time T between a tone of said first signal and the corresponding tone of said second signal;
variation of transit time ΔT as T-T₀.
variation of the propagation velocity of said pressure wave as the ratio between said distance of the arterial path comprised between the heart and the application point of said second sensor, and said variation of the transit time of the pressure wave.

This way, a differential value is obtained of the PWV between the basal and the post-basal conditions, which occurred after application of the stress. Not only, this differential value can be traced, during the effects of the stress, in order to have important responses, which can relate to the health conditions of the patient, and/or to its reactivity to suitable for drugs against hypertension or for reducing the cardiovascular risks.

Even if an instantaneous value of the PWV may not be very precise, which as above said is at least comparable to value obtainable with known systems, a differential value is, instead, of high precision, since possible measurement errors of the PWV, often owing to an inaccurate computing of the distance between the application point and the myocardium, are eliminated in the differential evaluation.

### BRIEF DESCRIPTION OF THE DRAWINGS

The invention will be made clearer with the following description of an exemplary embodiment thereof, exemplifying but not limitative, with reference to the attached drawings in which:

FIG. 1 shows a diagrammatical view of the application points of the first and of the second sensor of cutaneous vibrations on a patient;

FIG. 2 shows a block diagram that sums up the main functions of the apparatus, according to the invention, which is adapted to measure the propagation velocity of a pressure wave in a central arterial system;

FIG. 3 shows a time diagram that shows a plurality of signals relative respectively to the first and to the second sensor in addition to auxiliary signals, such as an ECG, an aortic pressure, a ventricular pressure, a pressure of the carotid artery and a diameter of the carotid artery;

FIG. 4 shows finally a block diagram that synthesizes the main functions relative to an apparatus, according to the invention, for measuring variation of the propagation velocity of a pressure wave in a cardiovascular system, in particular a pressure wave in a central arterial system;

FIG. 5 shows a diagrammatical view of a different arrangement of the first and of the second sensor of cutaneous vibrations located close to each other on the artery of a patient, in order to obtain a precise measurement of the pressure wave velocity,

finally, FIG. 6 shows a time diagram that shows two signals that are relative respectively to the first and to the second sensor applied to a same arterial vessel at a distance close to each other; the signals obtained are shifted temporally to each other.

### DETAILED DESCRIPTION OF SOME EXEMPLARY EMBODIMENTS

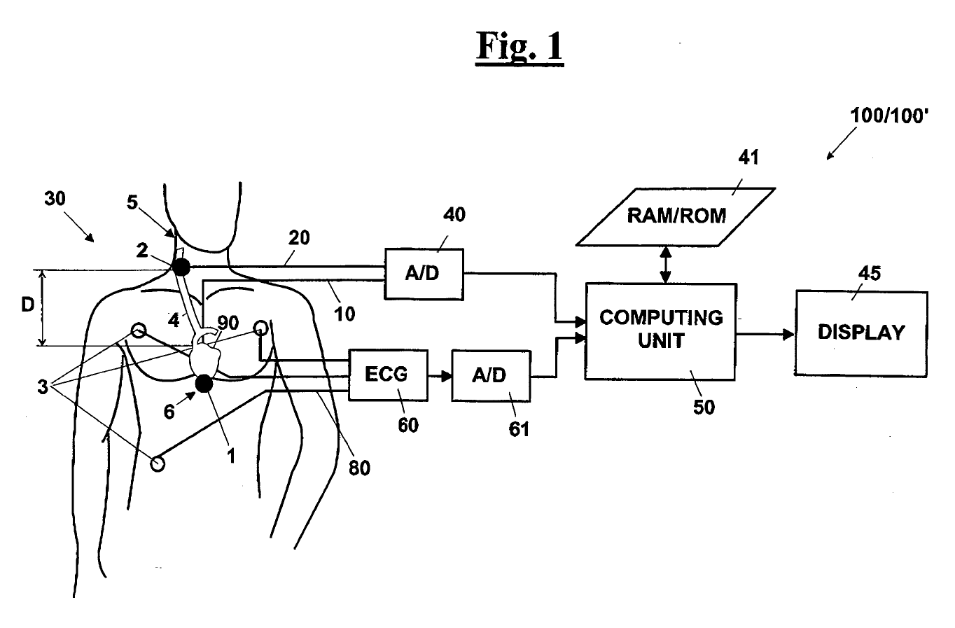

With reference to FIG. 1, a diagrammatical simplified view is shown of an apparatus 100/100′, according to the invention, for measuring the propagation velocity of a pressure wave. In particular, apparatus 100 comprises a first sensor of cutaneous vibrations 1 mounted in a first application point, in particular according to a first configuration, mounted at the heart, in order to measure a vibration generated by heartbeat 90, creating a corresponding first cutaneous vibration signal 10, and a second sensor 2, which is adapted to measure a local cutaneous vibration generated in a predetermined point of an arterial vessel 4, creating a corresponding second cutaneous vibration signal 20. More precisely, second sensor 2 detects the vibration caused by the deformation of vessel 4 responsive to the progression of the pressure wave. This way, it is possible to apply second sensor 2 for a long time on the patient's skin, at the application point of the sensor near arterial vessel 4, without any discomfort for the patient 30, and measuring continuously the second cutaneous vibration signal 20. The propagation velocity of the pressure wave, i.e. the PWV, is then calculated continuously, and a chart can be produced during diagnostic examinations such as, for example, stress tests. Furthermore, an instantaneous value of the PWV has a precision higher than that obtainable with known systems.

In fact, both sensors of vibration are applied in a light contact with the skin of the patient without applying any pressure. This is particularly relevant especially at the arterial vessel, since the adoption of cutaneous sensors makes it possible to eliminate measurement errors caused by the application of the sensor same. This way, in fact, it is avoided that, at the application point of the sensor, there is a restriction of the cross section and of the shape of the arterial vessel.

In particular, first sensor 1 for measuring the cutaneous vibrations generated by the heart is located on the sternum 6 of a patient 30 whereas second sensor 2 for acquisition of the signal of the cutaneous vibrations generated by arterial vessel 4 is located on the neck 5 of patient 30, being this vessel the carotid artery. Is, furthermore, necessary measuring the distance D of the application point of second sensor 2, near arterial vessel 4, with respect to the application point of first sensor 1. In particular, the distance D shown in the figure is shown in simplified way, since it is determined following the arterial path of vessel 4 and not tracing a conjunction line between heart 90 and application point 6 of sensor 2. In particular, the first 1 and second 2 sensors of cutaneous vibrations are adapted to measure vibrations set between 1 Hz and 20 KHz, in particular between 5 Hz and 80 Hz.

In particular, the first 1 and second 2 sensor of cutaneous vibrations are applied by means of sticking plasters, in order to provide a light contact with the skin of the patient at the selected application point. This way, they do not change the hemodynamics of the blood stream at their respective application point thus unaffecting the measurement of the pressure wave velocity.

The apparatus, furthermore, comprises a means for detecting the first 10 and second 20 cutaneous vibration signals as input towards a control unit 50. To this end, the signals 10/20 as input pass through an A/D converter 40.

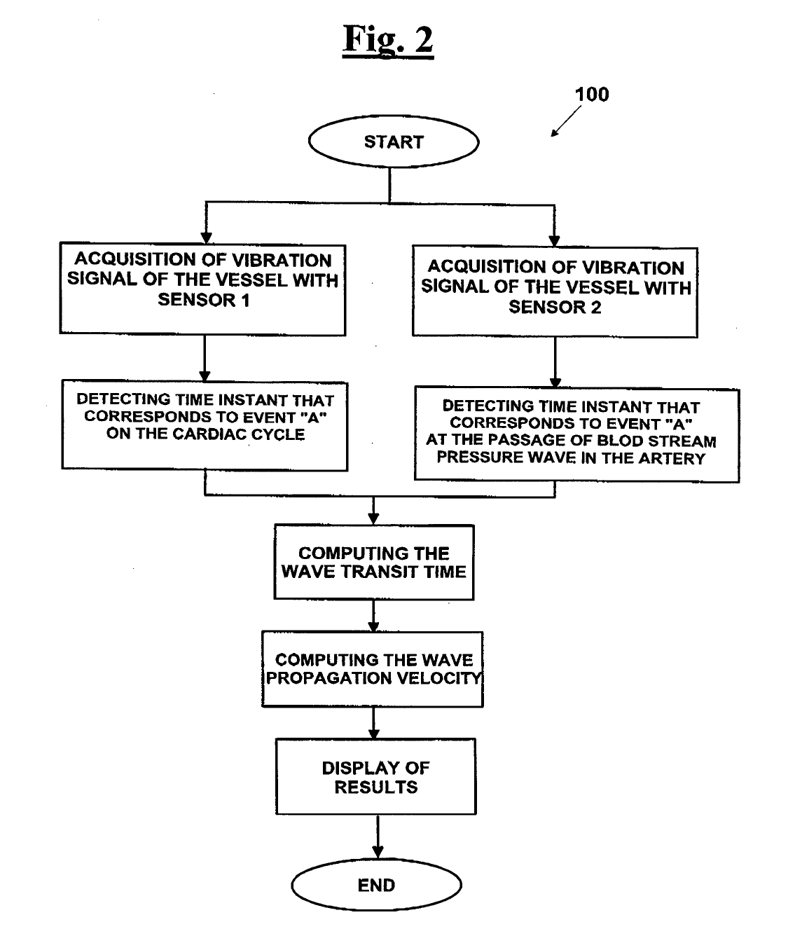

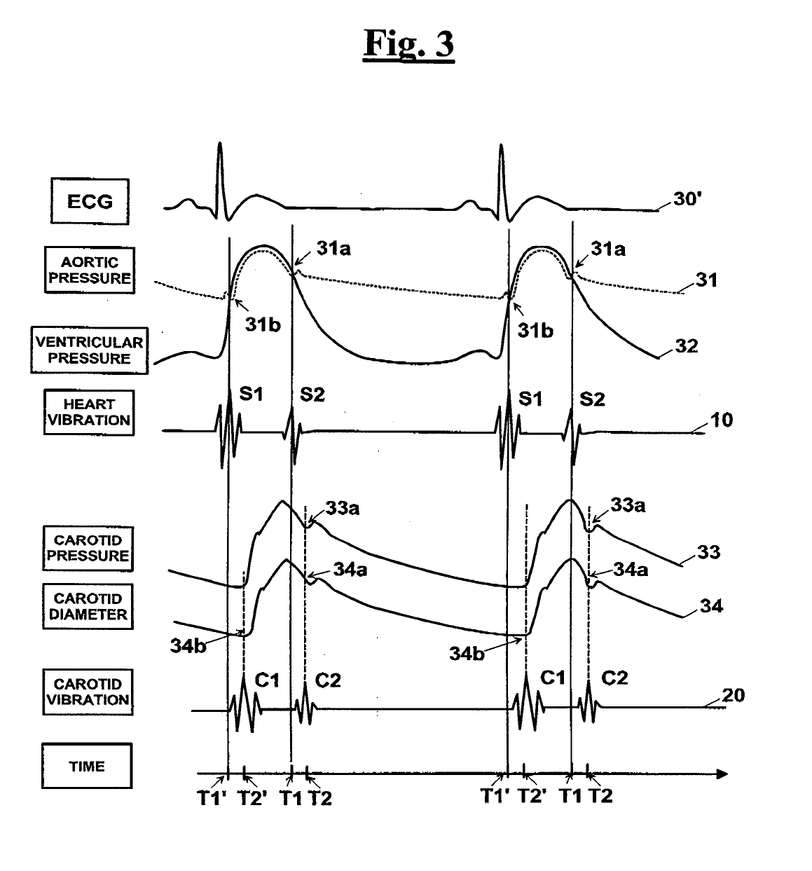

As diagrammatically shown in FIG. 2, in a block diagram that sums up the main functions of apparatus 100, after measurement of the two signals 10/20 control unit 50 computes the signals, by means of a dedicated program means, for calculating the propagation velocity of the pressure wave, responsive to the distance between the heart and the application point of second sensor 2 and to first 10 and second 20 cutaneous vibration signals. In particular, the program means determine respectively from first cutaneous vibration signal 10 a first time instant T1 (visible in FIG. 3) corresponding to a predetermined event of a cycle, and from second signal 20 a second time instant T2 (visible in FIG. 3) corresponding to the occurrence of the same event of the cardiac cycle as a local deformation of arterial vessel 4.

Control unit 50, calculates then a transit time PTT (Pulse Transit Time) of the pressure wave, as described below in detail, as a time period difference between first and second time instants T1 and T2 measured by respective sensors 1/2. Once determined transit time PTT, is then measured the propagation velocity of the pressure wave as the ratio between the length of the arterial path D comprised between the heart and application point 6 of second sensor 2 and said transit time PTT. The results thus obtained are displayed by a display. Once measured, the propagation velocity of the pressure wave can be used for calculating parameters of vascular stiffness, such as distensibility and Young modulus.

Advantageously, apparatus 100 can comprise, furthermore, sensors 3 for acquisition of an electrocardiographic signal 80 through an ECG device 60. Even in this case the signal 30 is encoded in a digital signal by an ND converter 61. This way, input signal 30 to control unit 50 can be used as time synchronism for determining the above described first T1 and second T2 time instants.

With reference to FIG. 3, a plurality of charts are given relative respectively to a elettocardiochart signal, chart 30′, an aortic signal called “aortic pressure” and shown by chart 31, as well as a corresponding ventricular signal called “ventricular pressure”, chart 32.

Furthermore, the chart depicts cutaneous vibration signal 10 determined by sensor 1 located on the sternum 6 of patient 30 and two respective charts relative to the pressure of the carotid artery, “carotid pressure”, chart 33, and to the carotid artery diameter, “carotid diameter”, chart 34. Finally, a signal 20 is determined by sensor 2 located at carotid artery 4 and by a reference time.

In particular, signal 10 of the vibrations of heart 90 has two peaks, the first at the beginning of the blood expulsion phase, at opening of aortic valve 31b, and the second at the end of the blood expulsion phase, i.e. at closing aortic valve 31a. These two peaks, if recorded in an audio band, correspond to first tone S1 and to second tone S2 of the phonocardiogram. In our case such peaks are referred to as first tone S1 and second tone S2, even if the considered signal has a band that is extended even outside the audio band, considering a cutaneous vibration signal characterised by a band extended towards below starting from the frequency zero.

In the same way, also signal 20 of the vibrations of arterial vessel 4 has two peaks; a first peak, referred to as C1, at the trough of wave 34b of the carotid artery diameter, and a second peak, referred to as C2, at the “Dicrotic Notch” 34a always of the carotid artery diameter. It is noted that such peaks are not obtained by the propagation along vessel 4 of sounds S1 and S2, but they are vibrations determined by the local deformation of the vessel same, which causes a corresponding cutaneous vibration.

Always as shown in FIG. 3, for determining the delay time PTT between the occurrence of a predetermined event of the cardiac cycle with respect to heart 90 and with respect to the distal point of vessel 4, namely in this case the point at sternum 6, it is necessary then to determine first time instant T1 that corresponds, for example, to closure of aortic valve 31a, as shown in chart 31, and second time instant T2 that corresponds to the so-called “Dicrotic Notch” 34a of the waveform of the diameter, shown by chart 34.

Alternatively, first time instant T1′ corresponds to opening aortic valve 31b whereas second time instant T2′ corresponds to beginning a quick increase of the diameter, i.e. the wave trough 34b. Such quick increase of diameter is due to arrival of the pulse pressure.

In the first case, the transit time PTT of the pressure wave, calculated by the analysis of the two generated signals of vibration 10/20, the former generated by the vibrations of heart 90 and the latter by the vibrations due to local deformation of the artery, in this case the carotid artery 4, is the difference between the instant of closure of aortic valve 31a defined on the heartbeat signal, chart 31, and the instant of the “Dicrotic Notch” 34a of the diameter defined on the vessel vibration signal, chart 34, since this “Dicrotic Notch” is just the occurrence of closing the aortic valve.

This way, with respect to the prior art, the present invention is different on how the occurrence of the cardiac event is determined with respect to arterial vessel 4. The principle is based on the fact that the pressure of the blood present into an arterial vessel generates locally a deformation of the vessel that causes a quick variation of diameter, which generates at a short distance a corresponding cutaneous vibration, as shown by chart 34. The waveform of the diameter can be assimilated to the waveform of the pressure, chart 33; these two chart are in phase with each other. It follows that the remarkable points of the waveform of the pressure, i.e. the trough of wave and the “dicrotic notch” 34a are present at a same time instant in both waveforms, as shown in FIG. 3. The movement of the vessel that follows the variation of diameter generates in turn of the vibrations that are measurable with the sensor of cutaneous vibrations 2, which is located on the patient's skin, near the vessel same. By the analysis of this signal as above described, it is therefore possible to determine the time instants corresponding to the remarkable points of the waveform of the diameter T2 and T2′, i.e. the remarkable points of the pressure waveform.

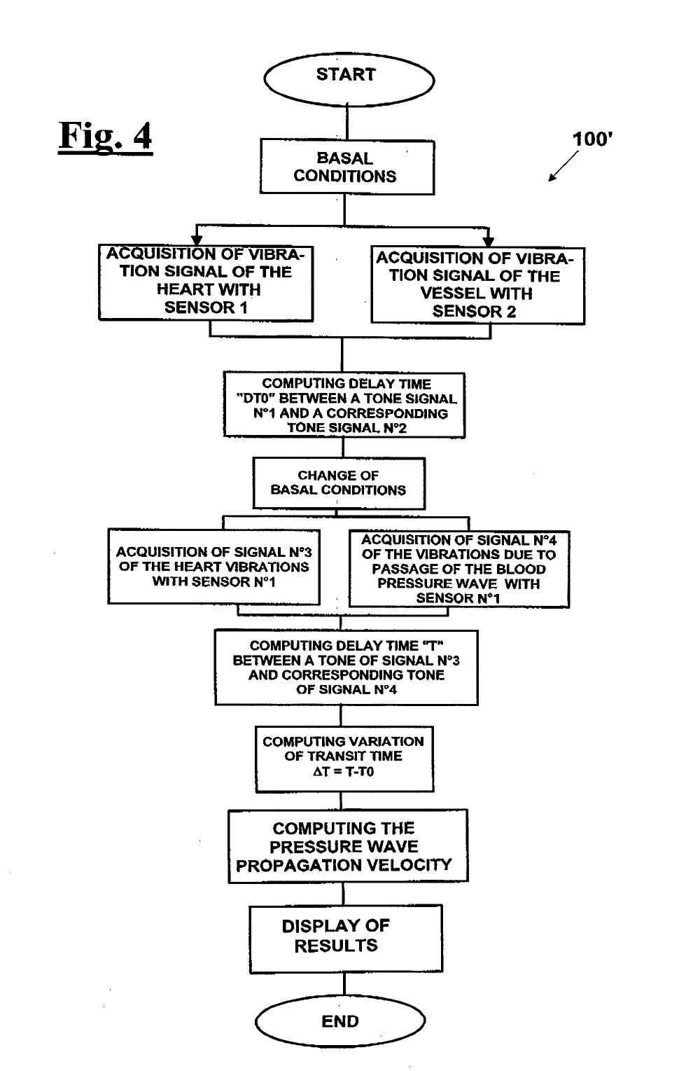

According to another aspect of the present invention, as shown in the block diagram of FIG. 4, in the field of diagnostic examinations such as, for example, pharmacological stress test and physical stress test, an apparatus 100′ is provide where the program means causes, in conditions that are prior to the imposition to the patient of a physical or pharmacological stress, or basal conditions, a delay time Tₒ between a tone of first signal 10 and a corresponding tone of second signal 20, and in conditions contemporaneous or next to the imposition to the patient of a physical or pharmacological stress, or post-basal conditions, delay time T between a tone of first signal 10 and the corresponding tone of second signal 20 and the variation of transit time ΔT as T-T₀.

On the basis of transit time ΔT the program means detect then variation of the propagation velocity of the pressure wave as the ratio between the distance of arterial path 4 comprised between heart 90 and application point of second sensor 2, and the variation of transit time ΔT of the pressure wave.

This way, a differential value is obtained of the PWV between the basal and the post-basal conditions, which occurred after application of the stress. Not only, this differential value can be traced, during the effects of the stress, in order to have important response, which can relate to the health conditions of the patient, and/or its reactivity to drugs against hypertension or for reducing the cardiovascular risks.

Even if instantaneous values of the PWV is of not high precision, which as above said is at least comparable to that obtainable with known systems, the differential value is, instead, of high precision, since possible measurement errors of the PWV, often owing to an inaccurate computing of the distance between the application point and the myocardium, are eliminated in the differential evaluation.

In particular, as shown in FIG. 3, apparatus 100′ detects the variation of transit time ΔT by measuring variation of the distance between the peaks of S1 and C1 and in a region of interest about them, or between the peaks of S2 and C2 and in a region of interest about them.

In particular, with respect to the previous case it is not any more relevant to determine precisely the event of opening or closing the aortic valve corresponding respectively at most on S1/C1 or S2/C2. In this case, in fact, the measure to carry out is a differential measure with respect to a basal condition.

Once measured variation of the propagation velocity of the pressure wave this data is used for calculating parameters of vascular stiffness during pharmacological stress test, physical stress test and stress test of other desired nature.

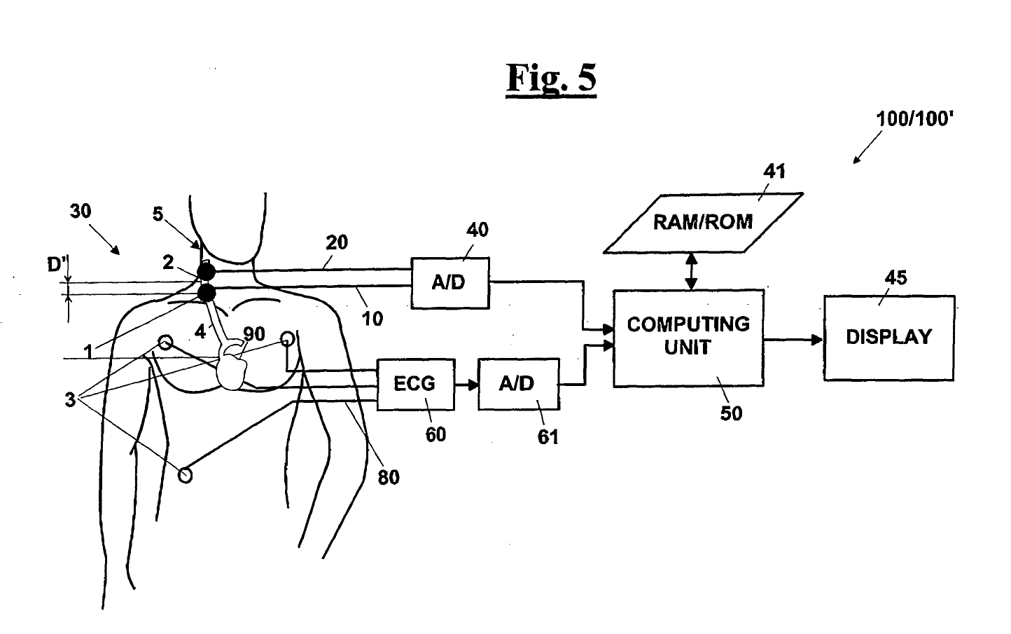

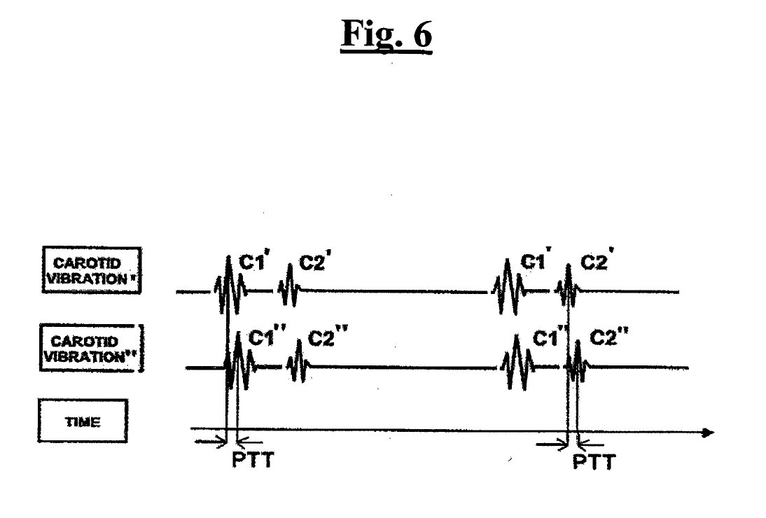

FIG. 5 shows another possible application of the apparatus that provides the application of the first and of the second sensor vibrations at a same arterial vessel, in particular of the carotid artery. In this case, the first and the second sensor of cutaneous vibrations are arranged at a distance OF close to each other, about several cm. This way, a precise measurement is obtained of the pressure wave velocity calculated considering two same signals of Carotid Vibration′ and Carotid Vibration″ (FIG. 6) delayed from each other for a range of time PTT that represents the (Pulse Transit Time). In this case, since it is the same arterial vessel, for example the carotid artery, the two signals of vibration are the same event of vibration slightly shifted temporally from each other, in order to give rise on the chart to two charts substantially equal to each other and shifted from each other on the axis time. This allows a high measurement precision, since in fact also the distance between the two points is measurable directly with precision. This allows a direct measurement of the PWV, with precision, particularly important for quick transients, for example by means of physical or pharmacological stress with quick response.

This local measuring type on the same vessel would be much more difficult or even impossible in case of systems according to the prior art, since with the traditional systems, a measurement of pressure at a short distance is substantially impossible. In fact, the known systems that use a pressure sensor would not allow a realistic measure, since they have to be applied using a predetermined pressure that would affect the flow of the blood. On the other hand, only the use of two sensors of cutaneous vibrations allows this detection since does not affect the hemodynamics of the arterial vessel and allow then to obtain a precise and reliable measurement.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realise the different functions described herein could have a different nature without, for this reason, departing from the field of the invention. It is to be understood that the phraseology or terminology employed herein is for the purpose of description and not of limitation.

1. An apparatus for measuring the propagation velocity of a pressure wave in a cardiovascular system, in particular a pressure wave arterial system, said apparatus comprising:
a first sensor of cutaneous vibrations, which is adapted to be mounted at a first application point of said arterial system for measuring a first cutaneous vibration, said first sensor creating a corresponding first cutaneous vibration signal;
a second sensor of cutaneous vibrations that is adapted to be mounted at a second application point of said arterial system different from said first point for measuring a second cutaneous vibration, said second sensor creating a corresponding second cutaneous vibration signal;
a means for determining the distance between said first and said second application points;
a control unit, said control unit comprising:
a means for detecting said first and said second cutaneous vibration signals as input towards said control unit;
a program means for calculating the propagation velocity of said pressure wave responsive to said distance between said first and said second application points and to said first and second cutaneous vibration signals,
wherein said first and second sensors of cutaneous vibrations are adapted to measure vibrations set between 1 Hz and 20 KHz, in particular between 5 Hz and 80 Hz.

2. An apparatus, according to claim 1, wherein said first sensor is adapted to be arranged in a first application point of the central arterial system and that is located at the heart, in order to measure the cutaneous vibration generated by the heartbeat, whereas said second sensor is adapted to be arranged at a second application point which is at an arterial vessel at a distance from the heart, in order to measure the vibration caused by the deformation of the vessel responsive to the movement of said pressure wave.

3. An apparatus, according to claim 1, wherein said first sensor is adapted to be arranged in a first application point of the central arterial system and that is located at an arterial vessel at a distance from the heart, whereas said second sensor is adapted to be arranged at a second application point which is at the same arterial vessel at a predetermined distance from said first point, in order to measure a local cutaneous vibration by the deformation of the vessel in each of said points located on said same arterial vessel.

4. An apparatus, according to claim 1, wherein said program means is adapted to determine:
on said first cutaneous vibration signal a first instant time corresponding to a predetermined event of a cardiac cycle;
on said second cutaneous vibration signal a second instant time corresponding to the occurrence of the same event of the cardiac cycle as a local deformation of said arterial vessel;
a transit time of said pressure wave as a time period difference between said first and said second instant times;
said propagation velocity of said pressure wave as the ratio between the distance between the heart and the application point of said second sensor and said transit time.

5. An apparatus, according to claim 1, wherein said first and second sensors are selected from the group comprised of: an accelerometer, a microphone, or an inertial sensor.

6. An apparatus, according to claim 1, wherein said first and second sensors of cutaneous vibrations are applied by means of sticking plasters or other adhesive means, in order to provide a light contact with the skin of the patient at said first and second application points.

7. An apparatus, according to claim 1, wherein said apparatus comprises acquisition means of an electrocardiographic signal, said electrocardiographic signal being used as synchronism time for determining said first and second instant times.

8. An apparatus, according to claim 1, wherein said first sensor for acquisition of the signal of the cutaneous vibrations generated by the heart is adapted to be located on the sternum whereas said second sensor for acquisition of the signal of the cutaneous vibrations generated by said arterial vessel is adapted to be located on the neck of the patient, in particular at the carotid artery.

9. An apparatus, according to claim 1, wherein said first instant time corresponds to closing the aortic valve whereas said second instant time corresponds to the “Dicrotic Notch” of the waveform of the vessel diameter vibration.

10. An apparatus, according to claim 1, wherein said first instant time corresponds to opening the aortic valve whereas said second instant time corresponds to start of a quick increase of the diameter, i.e. the wave trough.

11. An apparatus, according to claim 1, wherein said program means is adapted to cause:
in basal conditions delay time T₀ between a tone of said first signal and the corresponding tone of said second signal;
in post-basal conditions delay time T between a tone of said first signal and the corresponding tone of said second signal;
variation of transit time ΔT as T-T₀.
variation of the propagation velocity of said pressure wave as the ratio between said distance of the arterial path comprised between the heart and the application point of said second sensor, and said variation of the transit time ΔT of the pressure wave.
